(19) United States
(12) Patent Application Publication
(10) Pub. No.: US 2007/0232588 A1
(43) Pub. Date: Oct. 4, 2007

(54) Anti-parasitic and/or anti-viral and/or anti-microbial compositions

(76) Inventors: Leonard Stella, Pointe-Claire (CA); Soriba Cisse, Montreal (CA)

Correspondence Address: GEORGE A. SEABY;SEABY & ASSOCIATES, 250 CITY CENTRE AVNUE, OTTAWA, ON, K1R6K7, CA

(21) Appl. No.: 11/224,981

(22) Filed: Sep. 14, 2005

Related U.S. Application Data
(60) Provisional application No. 60/609,821, filed on Sep. 15, 2004.

(51) Int. Cl. A61K 31/55

(52) U.S. Cl. 514/214.03

(57) ABSTRACT

Voacamine and its natural and synthetic derivatives such as 16′-decarbometheoxyvoacamine, N₆-demethylvoacamine, voacamidine, tabernamine, ervahamine A, vobasine and coronaridine are useful as anti-microbial, anti-parasitic and anti-viral agents. A basic extract of the tertiary alkaloids present in plants of the genus Peschiera or Voacanga, especially Peschiera fuchsiaefolia can be isolated and used directly as such an agent.

### CROSS REFERENCE TO RELATED APPLICATION

This application claims priority on U.S. Provisional Application 60/609,821 filed Sep. 15, 2004.

### FIELD OF THE INVENTION

This invention relates to a method of treating a microbial, parasitic or viral infection using a basic extract of a plant of genus Peschiera or Voacanga.

### BACKGROND OF THE INVENTION

Protozoan parasites are responsible for some of the most important and prevalent diseases of humans and domestic animals, threatening the life of nearly one fourth of the human population. Such diseases are well known and include malaria (or paludism), leishmaniasis, trynomiasis, toxoplasma infections, protozoan intestinal infections, trychonomiasis giardiasis, isosporiasis, cryptosporidiosis, cyclosporiosis, microsporidiosis and the like. The World Health Organization (WHO) statistics show that amongst all parasitic infections due to protozoan, malaria and leishmania are the main cause of death in the world, with malaria being first and leishmania being in the second position. WHO statistics show that, with a 42-fold increase in the last 15 years, among protozoan parasitic diseases, leishmaniasis has become the second highest worldwide cause of death, just after malaria. In fact, leishmaniasis is endemic in 88 countries, with 350 million people at risk, 12 million affected by the disease, and 1.5-2 million new cases occurring annually. The disease is spreading in several areas because of massive rural-urban migration, drug resistance, and its association with AIDS. Leishmaniasis/HIV co-infection is indeed considered by WHO as a real threat, specially in Mediterranean countries and southwestern Europe. In addition, leishmaniasis is becoming endemic in the US.

Malaria is a severe endemic disease that afflicts the populations of tropical and sub-tropical zones and is carried by a mosquito. A recent report from WHO, shows that 300-500 million people suffer from this disease each year in the world with 70% of the cases occurring in sub-Sahara Africa and 4-6 cases/1000 in South America and southeast Asia. 1.5-3 million deaths occur every year, amongst which 1 million are children under 5 years . The parasite is a hematozoan of the Plasmodium genus such as Plasmodium vivax, Plasmodium ovale, Plasmodium malarae and Plasmodium falciparum, the latter being the most dangerous and devastating species. This disease is fought by various drugs, which are generally quinolitic alkaloids, such as chloroquine or aminocrinodines. Unfortunately, Plasmodia have gradually acquired a resistance to various, frequently used antimalarial drugs, in particular chloroquine, and such drugs have become inefficient or ineffective in many areas of the world. They must be replaced by other drugs such as the aminocrinodines, which unfortunately are toxic.

Leishmaniasis is a severe endemic disease which is carried by insects and prevails in 88 countries of the tropical and subtropical areas, putting at risk 350 million people and afflicting 12 million of them, with 1.5 to 2 million new declared cases each year. Leishmaniasis is caused by different species of the genus Leishmania, a kinetoplastid protozoan parasite from the family of Trypanosomatidae. These parasites present a digenetic life cycle, which include an infective flagellated promastigote form, present in the insect vector, and a non-motile intracellular form, the amastigote, that lives in the mononuclear phagocyte lineage of the host vertebrate.

The disease is spreading in several areas because of the massive rural-urban migrations, the emergence of drug resistance and its association with the AIDS. Leishmaniasis/HIV coinfection is indeed considered by WHO as a real threat, particularly in the Mediterranean and south-western Europe countries. Even the United States is on the way of becoming an endemic zone for Leishmaniasis.

The parasite is a Kinetoplastidae of Leishmania genus, for example Leishmania infantum, Leishmania tropica, Leishmania major, Leishmania mexicana, and especially, the most dangerous, Leishmania donovani.

Leishmaniasis is fought by various drugs, which are generally antimonial, such as N-methylglucamine antimoniate and sodium stibogluconate. Unfortunately, Leishmaniasis strains have developed a resistance against these drugs which have became inefficient in several areas of the world, in particular India where about 50% of the cases of visceral Leishmaniasis are resistant to the drugs. The substitute products to these drugs, such as pentamidine and amphotericin B, are unfortunately toxic and expensive. Even the new anti-leishmaniasis agents, which are alkyl-phospholipid analogues such as miltefosine which have given high hopes have already faced drug resistance in vitro.

In view of these facts,there is a need for new antiparasitic and antiviral agents to fight infectious diseases at a cost low enough to make them affordable to poor countries in which the diseases are prevalent.

### GENERAL DESCRIPTION OF THE INVENTION

The object of the present invention is to meet the above-defined need by providing an effective, relatively low cost method of treating microbial, parasitic and viral diseases.

Accordingly, the invention relates to a method of treating a microbial, parasitic or viral infection in a human or animal comprising the steps of exposing infected cells to a basic tertiary alkaloid extract of a plant of the genus Peschiera or Voacanga.

More specifically, the invention relates to a method as described above in which the basic alkaloids have the formulae: 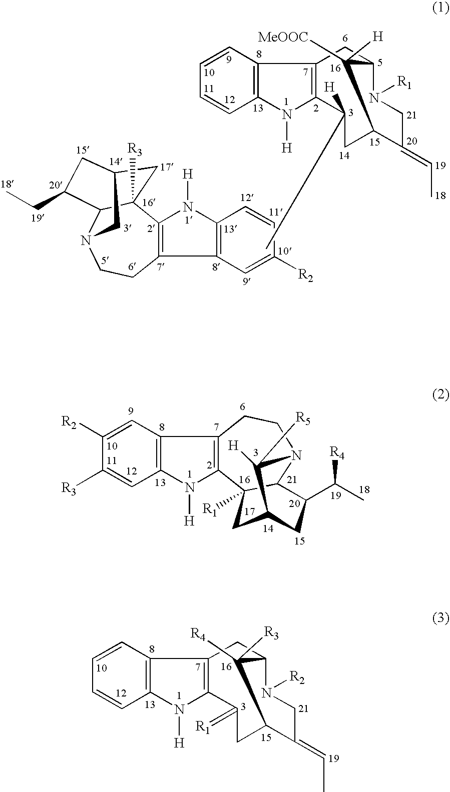
wherein R₁, is a methyl group or hydrogen, and R₂, R₃, R₄ and R₅, which are the same or different, are CH₂OH, CH₃, OCH₃, COOCH₃, OH or hydrogen.

The invention also relates to a method of isolating basic tertiary alkaloids from a plant of the genus Peschieraor Voacanga comprising the steps of pulverizing plant material of the genus Peschiera or Voacanga, treating the pulverized plant material with aqueous citric acid and Na₂HPO₄ buffer to yield an aqueous phase and an organic layer; extracting the organic layer with aqueous citric acid; adjusting the pH of the aqueous factions with a base; and extracting the basic tertiary from the aqueous fractions alkaloids with dichloromethane.

The invention is described below in greater detail with reference to the following examples and the accompanying drawings.

### BRIEF DESCRIPTION OF THE DRAWINGS

FIG. 1 is a graph showing the dose-dependent activity of alkoloid extracts from Peschiera fuchsiaefolia;

FIG. 2 is a graph showing the effect of basic alkaloid extracts from Peschiera fuchsiaefolia or Plasmodium falciparum growth;

FIG. 3 is a graph illustrating in vivo antiplasmodial activity of voacamine; and

FIG. 4 is a graph illustrating the in vitro activities of a basic extract from Peschiera fuchsiaefolia and purified voacamine against Leishmania infantum promastigotes.

### EXAMPLE 1

### Extraction

The starting material is the bark of the root of the Peschiera fuchsiaefolia harvested in Brazil, at Porto Alegre, which was identified locally from a botanical aspect by the pharmaceutical industry (Cibecol).

100 g of finely pulverized vegetal material were treated by extraction (3 times successively) with aqueous citric acid-Na₂HPO₄ buffer (14.0 g of Na₂HPO₄ and 10.0 g of citric acid/1 L of water) at a pH of 5. The aqueous fractions were discarded and the organic layer was extracted three times with 150 ml of an aqueous citric acid solution (3.1 g/L) at a pH of 3.5. The aqueous fractions were combined, the pH adjusted to 9.0 with Na₂CO₃ and extracted three times with 150 ml of dichloromethane. The organic fractions were combined, dried with anhydrous Na₂SO₄, filtered, and evaporated on a rotary evaporator to yield a residue of 3.4 g. This residue contains the tertiary alkaloids, which are the desired basic extract.

The activity of the basic extract was measured in vitro against Plasmodium falciparum (Example 4 below) and promastigote forms of a cloned Leishmania infantum (Example 6 below) strains grown at 28° C. in Roswell Park Memorial Institute (RPMI) 1640-modified medium supplemented with 20% heat-inactivated fetal bovine serum (both available from Gibco).

### EXAMPLE 2

### Purification

The diverse tertiary alkaloids contained in the basic extract of Example 1, were separated by countercurrent distribution, with dichloromethane as the stationary phase and an aqueous buffer with an incrementally decreasing pH (mobile phase). The alkaloids were recovered from the aqueous phase by extraction with dichloromethane.

The equipment used was a Craig type Post apparatus made up of 200 glass tubes (with 10 ml volumes for the lower phase and 10 ml volumes for the upper phase).

At pH 7, a first series of minor alkaloids were obtained, then at pH 5.2, there were eluted in order, perivine (KᵣK_{b}=4×10⁻⁹) 16-epi-affinine (KᵣK_{b}=2.5×10⁻⁹) and affinisine (KᵣK_{b}=7×10⁻¹⁰); where Kᵣ is the partition coefficient (aqueous phase/organic phase repartition) and K_{b} is the dissociation constant.

At pH 4, N-demethylvoacamine (KᵣK_{b}=3.5×10⁻¹¹) and vobasine (KᵣK_{b}=4×10⁻¹¹) were eluted.

At pH 3.2, voachalotine (KᵣK_{b}=2×10⁻¹¹) and voacamine (KᵣK_{b}=1.3×10⁻¹¹) were eluted.

At pH 3.0, heynearnine (KᵣK_{b}=5×10⁻¹²) and voacristine (KᵣK_{b}=3.5×10⁻¹²) were eluted.

At pH 2.6, conopharyngine (KᵣK_{b}=2×10⁻¹²) was eluted, and finally, at pH 2.2, voacangine (KᵣK_{b}=6.5×10⁻¹³) was eluted.

By countercurrent distribution in a biphasic system of dichloromethane, methanol and water 7/5/2 quaternary alkaloids, such as 12-methoxy-N₆-methylvoacalotine and N₆-methylaffinisine were obtained in chloride form. Each alkaloid was purified by one or many new recycling passages in a countercurrent distribution, and then by crystallization.

The pure alkaloid mixture was subjected to two additional chromatographic procedures to increase the purity; one on silica columns and the other on thin layer silica plates. The alkaloid mixture in a minimum volume of dichloromethane methanol (95:5) was then passed on to a 4.7×40 cm flash silica column. The alkaloids were eluted using the following methanol/dichloromethane gradient: 250 ml of 95:5; 300 ml of 90:10; 300 mL of 80:20; and finally, 300 ml of 60:40.

The compounds were then deposited on preparative thick, thin layer chromatography plates. Typically 100 mg were deposited per plate and migrated with 10% methanol in dichloromethane mixture. The alkaloids were UV visualized and the bands were scraped from the plates and eluted with 40% methanol in dichloromethane. The confirmation of the structure of the various alkaloids isolated was performed using ¹³C and ¹H NMR and mass spectroscopy.

### EXAMPLE 3

### Extract Analysis

The alkaloids in the basic extract were quantified using the following analytical method, which was validated. Calibration curves were constructed by dissolving purified alkaloids in a known amount of dichloromethane. A precise volume of the solution was taken, evaporated and diluted in a mixture containing 0.1% trifluoroacetic acid (TFA), 18% acetonitrile and 72% water. The samples were done in triplicate and analyzed by high performance liquid chromatography/mass spectrum (HPLC/MS) and the response factor determined using ions characteristic for each compound. These ions were 353 (M+2H)⁺², and 705 (M+H)⁺ for voacamine, 353 (M+H)⁺ for vobasine and 367 (M+H)⁺ for voachalotine. A calibration curve was performed for each of these ions at each concentration.

The residue crystallized from 300 mg of extract was dissolved in a mixture containing 0.1% TFA, 18% acetonitrile and 72% water. The mixture was sonicated 5 minutes, passed on a 0.2 μm polytetrafluoroethylene (PTFE) filter, and 50 pi of the solution injected. The analyses were done in triplicate. Total ion chromatogram demonstrated the intensity of the ions corresponding to the pseudomolecular ions (M+H)⁺ of the alkaloid standards, namely 705 for voacamine (retention time 29.7 s) and its isomer (retention time 28.8 s), vobasine (retention time 16.1 s) and voachalotine (retention time 20.6 s). These ions were used to calculate the concentration of each compound in the extract using the response factors obtained from the calibration curve.

TABLE 1
Relative intensities of selected ions corresponding
to monomeric alkaloids versus the total intensities
of the ions chromatograms of the plant extract.

| | | Retention | |
|---|---|---|---|
| | Ion | time | Percentage |
| Alkaloid | monitored | (min) | in extract |
| Affinisine^{a} | 306 | 4.75 | ND |
| Affinisine^{a} | 306 | 21.9 | 0.0 |
| Tabernanthine | 311 | 21.5 | 1.1 |
| 16-epi-affinine | 325 | 14.5 | 1.5 |
| Heynearnine^{a} | 355 | 17 | 0.4 |
| Heynearnine^{a} | 355 | 21.2 | 0.0 |
| Perivine | 339 | 24.5 | 4.4 |
| Voacangine | 369 | 23.1 | 5.6 |
| Voacristine | 385 | 19.9 | 0.1 |
| Conopharyngine | 399 | 18.7 | 0.3 |
| 1 2-methoxy-N₆-methyl Voacalotine | 411 | 24.6 | 1.2 |

^{a}Due to the absence of authentic standards, these compounds could not positively be identified in the mixtures. They are possible structures corresponding to the ions selected. ND: Not Detected
The ions selected correspond to the pseudomolecular ions of various alkaloids known to be present in this plant.

TABLE 2
Relative intensities of selected ions corresponding
to dimeric alkaloids versus the total intensities
of the ions chromatograms of the plant extract.

| | | Retention | |
|---|---|---|---|
| | Ion | time | Percentage |
| Alkaloid^{a} | monitored | (min) | in extract |
| Vobasine | 353 | 16.1 | 0.120 |
| Voachalotine | 367 | 20.6 | 0.557 |
| Tabernamine | 617 | 24.7 | 0.017 |
| 16-Decarboxy- | 647 | 27.5 | 0.13 |
| Methoxyvoacamine | | | |
| Ervahanine A | 675 | 27.5 | 0.018 |
| N₆-demethylvoacamine | 691 | 26.9 | 0.18 |
| Voacamine isomer^{b} | 705 | 28.8 | 0.231 |
| Voacamine^{b} | 705 | 29.7 | 0.779 |

^{a}The dimeric alkaloids of the root bark are thought to be responsible for the biological activity of the mixture, like voacamine.
^{b}Due to the absence of authentic standards, these compounds could not positively be identified in the mixtures. They are possible structures corresponding to the ions selected.

### EXAMPLE 4

### In Vitro Antiplasmodial Activity

The basic extracts obtained as in Example 1, from both the root and stem bark with a yield of 1.9% were used to carry out the in vitro antiplasmodial activity study on known Plasmodium falciparum strains. The results obtained are reported in Tables 3 and 4 below.

Plasmodium falciparum strains D₆ and W₂ were used throughout this investigation. Laboratory isolates of P. falciparum were grown under controlled conditions in culture medium containing leukocyte-free red blood cells (RBCs) at 5% hematocrit (Tager and Jensen, 1976). Briefly, parasites were allowed to infect RBCs in a media consisting of RPMI 1640 plus 25 mM Hepes, 0.25 glucose, 0.2% sodium bicarbonate, 0.5% Albumax II, and 50 mg/liter hypoxanthine and grown in 5% CO₂ at 37° C. When required, cultures were synchronized by sorbitol treatment (D. Ramanitrahasimbola et al, 1999, Biological Activities of the Plant-derived Bisindole Voacamine with reference to Malaria. Phytother. Res. 15: 30-33).

Assessment of parasite development and morphology. Parasite development and morphology were evaluated by microscopic examination of Giemsa-stained thin blood smears at 24, 48, and 72 hour intervals in the presence of increasing doses of the alkaloids dissolved as a stock solution in 10% (v/v) dimethylsulphoxide (DMSO) in RPMI 1640. Dilutions of stock solutions were prepared in RPMI 1640 medium before use. Smears from drug-free cultures were always used as controls Parasitemia was measured by counting blood cells and expressed as percentage of total parasitized erythrocytes (Table 3).

TABLE 3
In vitro activities of basic extract from
Peschiera fuchsiaefolia and purified voacamine
against Plasmodium falciparum

| IC₅₀ | Strain D₆ | Strain W₂ |
|---|---|---|
| Ex. 1 | 179 | 282 |
| Ex. 2 | 238 | 290 |
| Ex. 3 | 495 | 817 |

The values are expressed in ng/ml. The lower they are, the more active the product. Voacamine purified from the total plant extract had remarkable anti- plasmodial activity against the two types of strains (Ex.2). Its activity is similar to that of chloroquine against sensitive D₆. The activity of the basic extract from the stem bark (Ex.3) was less potent, but it still retained its activity against the chloroquine resistant strains of Plasmodium W₂. The most potent compounds are the alkaloids contained in the basic extract from the root bark (Ex.1).

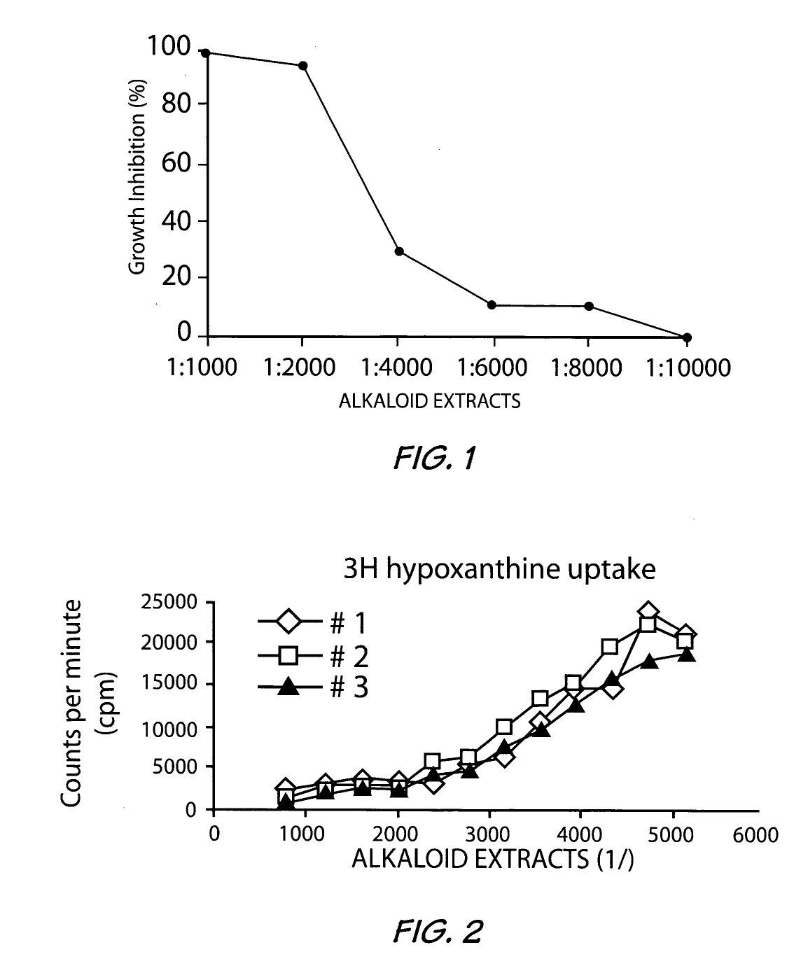

As illustrated in FIG. 1, in the presence of the extract compounds, there was a dose-dependent growth inhibition against the parasites.

TABLE 4
In vitro inhibition of Plasmodium falciparum
growth by the Basic Alkaloid Extracts
from Peschiera fuchsiaefolia

| Alkaloid | | Growth | |
|---|---|---|---|
| Extract | Mean cpm | inhibition | Parasite morphology |
| dilution | (±S.D.)^{a} | (%) | Giemsa stain (48 hrs) |
| 1:1000 | 926 ± 382 | 99 | Destruction of parasites |
| 1:2000 | 1904 ± 204 | 94 | Destruction of parasites |
| 1:4000 | 13694 ± 4092 | 30 | Destruction of parasites |
| 1:6000 | 17320 ± 364 | 11 | Vacuolation and Pycknosis |
| 1:8000 | 17329 ± 1669 | 11 | Vacuolation and Pycknosis |
| 1:10000 | 22910 ± 1156 | 0 | Vacuolation and Pycknosis |

^{a}Data are reported as mean and standard deviation (S.D.) of percentage parasite growth inhibition in triplicate experiments relative to untreated controls after correcting for background ³H-hypoxanthine incorporation in uninfected erythrocytes.

Qualitative microscopic examination of the blood smears demonstrated a strong inhibitory effect of the alkaloid extracts on parasite development after 24 hrs. There was a dramatic alteration of normal morphology with the most prominent changes being pycknosis and vacuolation (changes indicative of cellular destruction). After 48 hours (a single sexual cycle), there was complete degeneration and destruction of parasites and their host cells.

Growth inhibition assay. The growth inhibition of the parasites was assessed by subsequently treating the cultures with a series of various dilutions of the basic extracts of the alkaloids from P fuchsiaefolia followed by serial two-fold dilutions in complete modified medium to contain 2.5 mg/liter hypoxanthine (low hypoxanthine medium). The dilutions were added to 96-well culture microplates at 100 μl/well. Parasites were diluted to a 2-fold concentrated stock solution containing 1-2% parasitemia and 5% hematocrit in low hypoxanthine medium and this suspension was added at 100μl/well. The activity of the extracts was evaluated after 24 hour intervals.

Parasite replication was assessed using the ³H-hypoxanthine assay. Parasites use hypoxanthine included in the growth media as a precursor in nucleic acid synthesis. By replacing hypoxanthine in the media with radioactive hypoxanthine, the rate of DNA replication and growth rate of the parasites in the presence of antimalarial drugs can be measured.

Hypoxanthine assays were performed on parasites in the presence of different concentrations of the alkaloid extract. After 24 hour incubation, 100 μl of culture supernatants were replaced with 100 μl of low hypoxanthine medium containing ³H-hypoxanthine at a final concentration of 0.5 μCi (35). After an additional 18 hours, supernatants were removed and cells harvested onto glass fiber filters. Air-dried filters were immersed in scintillation fluid and radioactive emissions were counted in a liquid scintillation counter machine.

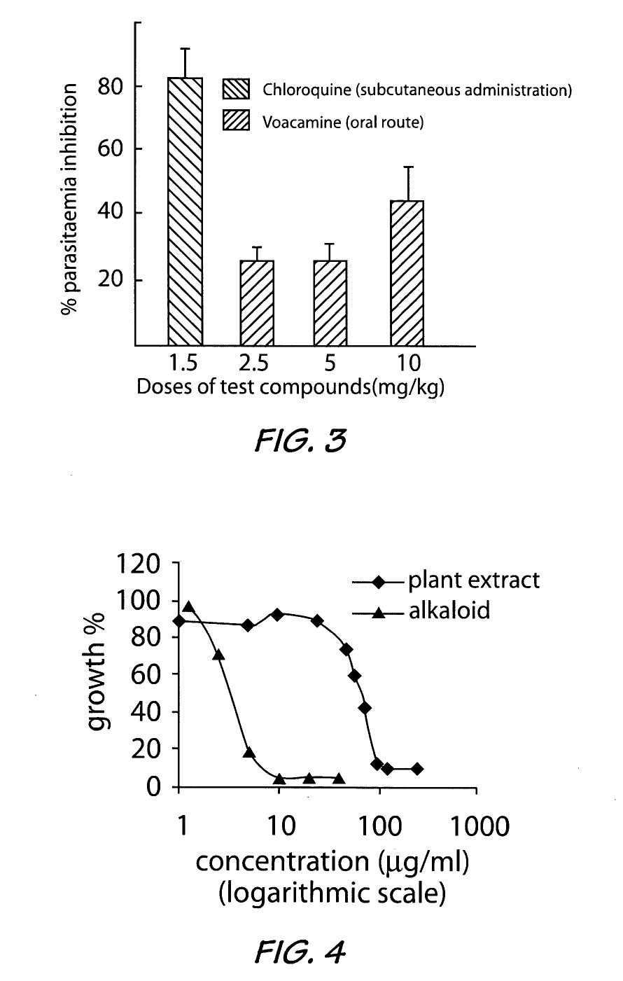

Control RBCs infected with parasites in the absence of the alkaloid extract incorporated radioactivity to a level of 50000 counts per minute (cpm). In the presence of alkaloid extract, there was a dramatic decrease in ³H-hypoxanthine uptake, indicating a substantial decrease in parasite replication and growth. In fact, a dilution of the alkaloid extract as low as 1/1200 caused a greater than 97% inhibition of ³H-hypoxanthine uptake of 3000 cpm (FIG. 3). The effect of drug was also dose dependant, and demonstrated greater than 50% inhibition at dilutions greater than 1/500 (20900 cpm).

Assays were performed in triplicate and the mean percentage growth inhibition was plotted as a function of the extracts concentration. Growth inhibition (%) was calculated from the counts per minute (cpm) derived from the ³H-hypoxanthine uptake assays.

### EXAMPLE 5

### In Vivo Antiplasmodial Activity

Animal model. Assessment of blood schizoncidal activity was performed following the classical 4 day suppressive test (D. Ramanitrahasimbola et al, supra), using Plasmodium yoelii N67 as rodent malaria parasite and chloroquine as reference antimalarial. Donor Swiss albinos mice weighing 20±2 g were inoculated via the tail vein with 10⁷ red blood cells parasitized with the rodent malaria parasite obtained from the donors. Three groups were then treated once daily, starting from the day of the test (day 0) respectively with 2.5, 5 and 10 mg/kg of voacamine citrate in 0.9% NaCl water solution by oral route. The fourth group was treated with 1.5 mg/kg of chloroquine sulfate in 0.9% NaCl water solution by subcutaneous administration while the controls (fifth group) received the vehicle. Treatments were repeated on days 1, 2, and 3. On day 4, the percentage of parasitized red blood cells was determined microscopically on 2000 red blood cells using blood smears obtained from the tail and stained with Diff Quick reagent. The percentage parasitemia of all mice in each group was recorded and the mean percentage parasitemia for each group was calculated by common statistical procedures and compared with that observed in the untreated controls. Tests were performed in three independent experiments and the results are presented in the form of histogram (FIG. 4). At the dose levels used, voacamine, one of the active principles of the basic alkaloid extracts from Peschiera fuchsiaefolia showed significant in vivo antiplasmodial activity in the 4-day test.

Clinical assay. Clinical trials were carried in a group of 74 malaria patients, in Mozambique, an endemic zone of chloroquine resistant strains of Plasmodium.

Each of the patients was injected in the 7^{th,} 8^{th} or 13^{th} vertebra, with 4 ml of the basic extract of example 1 diluted in 100 ml of physiological saline solution.

After 3 hours, the clinical signs of malaria (fever, vomiting, diarrhea, joint pains) were cleared in most of the cases and after 4 hours, all red blood cell cultures from these patients were negative.

In 4 days 72% of the patients were cured, and in the following 3 consecutive days during the treatment, 90% were cured.

Some of patients were administered orally (syrups) and others by anal route in order to detect possible undesirable side effects. No side effects were observed as compared to the main drugs currently used in the fight against malaria, which showed toxic side effects in endemic areas of multidrug resistant malaria.

### EXAMPLE 6

### In Vitro Leishmanicidal Activity

Preparation of the anti-leishmanial compositions: The total plant extract was dissolved in DMSO as a 50 mg/mL stock. The purified alkaloid, voacamine, was dissolved in 50% DMSO at a 3 mg/mL stock.

In vitro drug testing. The in vitro leishmanicidal effect of purified voaca mine against Leishmania infantum promastigotes was evaluated by the calorimetric 3-(4,5-dimethyl-2-thiazolyl-2,5-diphenyl-2H-tetrazoliumbromide (MTT) based assay. The screening was performed on promastigote forms from a logarithmic phase culture suspended in fresh medium. 2×10⁶ parasites/mL were seeded in 96-well microtiter plates, with increasing concentrations of the compounds to a final volume of 150 μL. The final DMSO content did not exceed 0.3%, which had no effect on parasite growth. After 72 hours of incubation at 28° C., parasite morphology was examined under a microscope, and the growth and viability of promastigotes was determined by the colorimetric MTT assay. 10 μL of 3-(4,5-dimethylthiazol-2-yl)-2,5-diphenyl-2H-tetrazol bromide (MTT, Sigma, 5 mg/mL in PBS) were added to each well and plates were incubated for an additional period of 5 hours. Only viable cells are able to reduce the MTT compound to produce water-insoluble formazan crystals of a purple color. These crystals were dissolved by adding 100 μL of SDS 20% and incubating overnight at 37° C. The absorbance was subsequently read at 540 nanometers using a microplate reader (Beckman Biomek 2000). Cell survival was determined by dividing the absorbance of the compound at a given concentration by the absorbance of control cells grown in the absence of drug. The 50% inhibitory concentration (IC₅₀) values were graphically determined after plotting the percentage of growth as a function of drug concentration. All experiments were performed in duplicate.

The results are depicted in FIG. 4 and Table 5 below. The values for the IC₅₀ (the concentration of compound required to kill 50% of the parasites) are expressed in ng/ml. The lower the IC₅₀, the more effective the compound. In the presence of the Peschiera fuchsiaefolia basic extract, there was a dose-dependent growth inhibition, with an IC₅₀ of only 68.2 ng/ml against Leishmania infantum promastigotes. Complete growth inhibition was observed at concentrations higher than 100 μg/ml.

The alkaloid voacamine, purified from the basic plant extract, had a higher in vitro leishmanicidal effect against promastigotes. Concentrations as low as 10 (μg/ml were able to produce complete cellular destruction before 72 hours and the kill curves showed an IC₅₀ of only 3.3 ng/ml, which means a 20-fold increase with respect to the basic extract.

TABLE 5
In vitro activities of the basic extract from
Peschiera fuchsiaefolia and purified voacamine
against Leishmania infantum promastigotes.

| | Plant extract | Voacamine | Fold Increase |
|---|---|---|---|
| 1C₅₀(ng/mL) | 68.2 | 2.3 | 20.7 |
| IC₉₀(ng/mL) | 110 | 7 | 15.7 |
| n^{a} | 3 | | 1 |

^{a}Number of experiments, each in duplicate.

1. A method of treating a microbial, parasitic or viral infection in a human or animal comprising the steps of exposing infected cells to a basic tertiary alkaloid extract of a plant of the genus Peschiera or Voacanga.

2. The method of claim 1, wherein said plant is Peschiera fuchsiaefolia.

3. The method of claim 1 wherein said basic extract contains alkaloids having the formula selected from the group consisting of 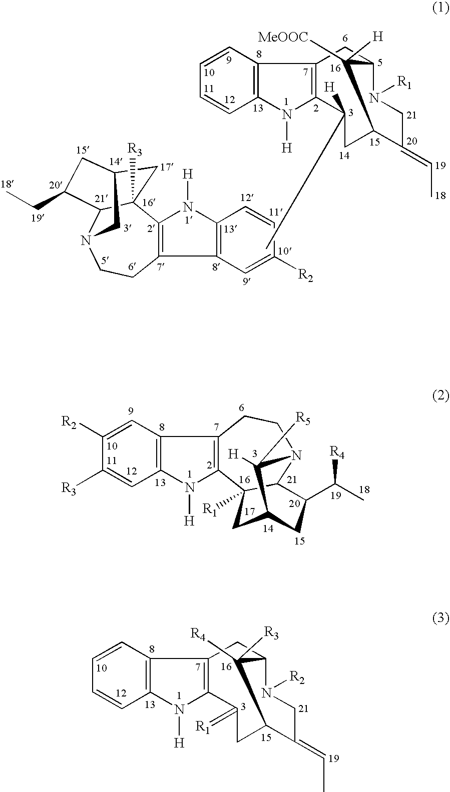
wherein R₁ is a methyl group or hydrogen, and R₂, R₃, R₄ and R₅, which are the same or different, are CH₂OH, CH₃, OCH₃, COOCH₃, OH or hydrogen.

4. A method of isolating basic tertiary alkaloids from a plant of the genus Peschiera or Voacanga comprising the steps of pulverizing plant material of the genus Peschiera or Voacanga, treating the pulverized plant material with aqueous citric acid and Na₂HPO₄ buffer to yield an aqueous phase and an organic layer; extracting the organic layer with aqueous citric acid; adjusting the pH of the aqueous factions with a base; and extracting the basic tertiary from the aqueous fractions alkaloids with dichloromethane.

5. The method of claim 4, wherein said plant is Peschiera fuchsiaefolia.

6. The method of claim 5, wherein the only root of said Peschiera fuchsiaefolia is used to obtain the basic tertiary alkaloid extracts.
